(19) United States
(12) Patent Application Publication
(10) Pub. No.: US 2018/0153683 A1
(43) Pub. Date: Jun. 7, 2018

(54) IOL WITH REDUCED PUPILLARY REFLECTIONS

(71) Applicant: AMO GRONINGEN B.V., Groningen (NL)

(72) Inventors: Robert Rosén, Groningen (NL); Mihai State, Groningen (NL)

(21) Appl. No.: 15/828,095

(22) Filed: Nov. 30, 2017

Related U.S. Application Data
(60) Provisional application No. 62/428,438, filed on Nov. 30, 2016.

(51) Int. Cl. A61F 2/16

(52) U.S. Cl. CPC A61F 2/1613; A61F 2002/1696

(57) ABSTRACT

An apparatus, system or method for providing an intraocular lens that reduces pupillary reflections. The apparatus or system may include a set of intraocular lenses configured to provide an optical power between about 5 Diopter and about 34 Diopter at a predefined increment there between, each lens having a shape factor configured such that the magnitude of intensity of light reflected from any intraocular lens is within two orders of magnitude of the intensity of light reflected from any other lens in the set. The method for designing an intraocular lens may include obtaining physical or optical characteristics of a patient's eye and then determining a shape factor of an intraocular lens by selecting a value for a radius of curvature of a surface of the intraocular lens to reduce a peak intensity of reflected ambient light over a range of clinical optical powers.

### CROSS-REFERENCE AND RELATED APPLICATIONS

This application claims priority to, and the benefit of, under U.S.C. § 119(e) of U.S. Provisional Appl. No. 62/428,438, filed on Nov. 30, 2016, which is incorporated herein by reference in its entirety.

### BACKGROUND

### Field

This disclosure generally relates to devices, systems and methods that reduce pupillary reflections.

### Description of Related Art

Intraocular Lenses (IOLs) may be used to restore visual performance after a cataract or other ophthalmic procedure in which the natural crystalline lens is replaced with or supplemented by implantation of an IOL. Some patients with implanted IOLs report reflections from the anterior surface of the IOL which can be seen by other people facing the patient or by the patient in a mirror. These reflections can be disconcerting for some patients.

### SUMMARY

The systems, methods and devices of the disclosure each have several innovative aspects, no single one of which is solely responsible for the desirable attributes disclosed herein.

Current IOL technologies are configured to correct refractive errors at the fovea. However, ambient light can be reflected from the anterior surface of various IOLs currently available in the market. Such reflections are referred to herein as pupillary reflections. The pupillary reflections can give the appearance of “glowing eyes,” or “twinkling eyes” which maybe disconcerting to patients. Embodiments of IOLs discussed herein are configured to reduce reflections from the anterior surface of the IOLs. For example, the radius of curvature of an anterior surface that receives ambient light refracted by the cornea of various embodiments of IOLs discussed herein can be configured to reduce intensity of reflected ambient light. The embodiments of pupillary reflections reducing IOLs discussed herein may or may not be configured to improve peripheral image quality.

Various systems, methods and devices disclosed herein are directed towards intraocular lenses (IOLs) including, for example, posterior chamber IOLs, phakic IOLs and piggyback IOLs, which are configured to have reduced pupillary reflections. Various embodiments of the pupillary reducing IOLs described herein can have an anterior radius of curvature greater than 42 mm or less than 19 mm. It is found that the intensity of reflected light from anterior surface of embodiments of IOL having an anterior radius of curvature greater than or equal to about 42 mm and less than or equal to about 19 mm have reduced reflections as compared to light reflected light from anterior surface of embodiments of IOL having an anterior radius of curvature greater than about 19 mm and less than about 42 mm, especially between about 24 mm and about 30 mm is greater than the intensity of reflected light from anterior surface of embodiments of IOL having an anterior radius of curvature greater than about 30 mm and/or less than about 24 mm.

### BRIEF DESCRIPTION OF THE DRAWINGS

The systems, methods and devices may be better understood from the following detailed description when read in conjunction with the accompanying schematic drawings, which are for illustrative purposes only. The drawings include the following figures:

FIG. 1 illustrates the variation of light reflected from an anterior surface of an embodiment IOL as a function of radius of curvature of the anterior surface.

FIG. 2 illustrates the variation of light reflected from an anterior surface of an embodiment IOL as a function of radius of curvature of the anterior surface when the embodiment of the IOL is placed in a physical eye model and the variation of light reflected from an anterior surface of an embodiment IOL as a function of radius of curvature of the anterior surface when the embodiment of the IOL is placed in a real eye.

FIG. 3 illustrates the variation in the intensity of light reflected from the anterior surface having a radius of curvature of 26 mm as a function of the effective lens position (ELP).

FIG. 4 illustrates the variation in the intensity of light reflected from the anterior surface having a radius of curvature of 26 mm as a function of the position of an observer.

FIG. 5 illustrates the variation in the intensity of light reflected from the anterior surface having a radius of curvature of 26 mm as a function of the wavelength of incident light.

FIG. 6 illustrates the variation in the intensity of light reflected from the anterior surface having a radius of curvature of 26 mm as a function of the corneal radius.

FIG. 7A is a photograph depicting the reflection of light from an IOL having an anterior surface with a radius of curvature of about 1000 mm placed in the physical eye model. FIG. 7B is a photograph depicting the reflection of light from an IOL having an anterior surface with a radius of curvature of about 26 mm. FIG. 7C is a photograph depicting the reflection of light from an IOL having an anterior surface with a radius of curvature of about 12 mm.

FIG. 8A is a photograph depicting the reflection of light from a first embodiment of a commercially available IOL. FIG. 8B is a photograph depicting the reflection of light from a second embodiment of a commercially available IOL. FIG. 8C is a photograph depicting the reflection of light from a third embodiment of a commercially available IOL.

FIG. 9A is a graph of the intensity of light reflected from various embodiments of IOLs having different shape factors and comprising a material having refractive index of 1.47 for different optical powers.

FIG. 9B is a graph of the intensity of light reflected from various embodiments of IOLs having different shape factors and comprising a material having refractive index of 1.52 for different optical powers.

FIG. 9C is a graph of the intensity of light reflected from various embodiments of IOLs having different shape factors and comprising a material having refractive index of 1.55 for different optical powers.

FIG. 10 is a flow chart of a method of designing an IOL that reduces pupillary reflection.

FIG. 11 is a graphical representation of the elements of computing system for designing an IOL.

### DETAILED DESCRIPTION

The phenomenon of glowing eyes has been observed in several animals. For example, the glowing eyes of a cat are apparent in night time photographs of a cat. The glowing eyes of the cat can be attributed to the highly reflectivity of the cat's retina. The human retina however, is not as reflective as the cat's retina and thus most phakic eyes that have the natural crystalline lens do not exhibit intense light reflections. However, some patients who have undergone ophthalmic procedures (for example, due to cataract or some other eye diseases) and have been implanted with an intraocular lens report glowing or twinkling eyes which can be perceived by an observer facing these patients or by the patients themselves in a mirror or some other reflecting surface. The glow or twinkle in pseudophakic eyes (eyes with IOLs) can be at least partially attributed to reflection of ambient incident light from the anterior surface of the IOL which received light reflected by the cornea. Such reflections are referred to herein as pupillary reflections. These pupillary reflections can be disconcerting/disturbing to patients and be undesirable from a cosmetic point of view. Thus, it would be advantageous to reduce the intensity of pupillary reflections

The intensity of pupillary reflections can depend on a variety of factors including but not limited to refractive index of material of the IOL and the geometry (e.g., curvature, sag, etc.) of the surfaces of the IOL. For example, in accordance with Fresnel's equations, the reflectivity coefficient for light incident from a medium having an index of refraction nₘ on a medium having an index of refraction n₁ at an angle of incidence of 0 degrees (i.e. substantially along the normal to the medium having the index of refraction n₁) is given by the following equation (1):

$$R = {( \frac{( {n}_{1} - {n}_{m}}{( {n}_{1} + {n}_{m} )} )}^{2} \qquad ( 1 )$$

Thus, an IOL comprising a material having a refractive index value of 1.47 (e.g., SENSAR®) when implanted in the eye and surrounded by the aqueous humor having an index of refraction of 1.33 will have a reflectivity coefficient of about 0.25% for light incident on the IOL along a direction substantially parallel to the optical axis of the eye, while an IOL comprising a material having a refractive index value of 1.55 (e.g., ACRYSOF®) when implanted in the eye and surrounded by the aqueous humor having an index of refraction of 1.33 will have a reflectivity coefficient of about 0.58% for light incident on the IOL along a direction substantially parallel to the optical axis of the eye. Accordingly, an IOL comprising a material having a refractive index value of 1.47 (e.g., SENSAR®) can have reduced pupillary reflection intensity as compared to an IOL comprising a material having a refractive index value of 1.55 (e.g., ACRYSOF®).

The effect of the geometry of the surfaces of the IOL on intensity of pupillary reflections is studied herein. Accordingly, a simulation tool is constructed to estimate the reflectivity for various IOL geometries. The simulation tool comprises: (1) an element representing a light source configured to provide a collimated light beam or a converging light beam emitted from a distance 1 m away that simulates a vergence of 1 Diopter; (2) an element representing an anterior surface of the cornea having a radius of curvature between 6 mm and 10 mm configured to refract light from the element representing the light source; (3) an element representing a posterior surface of the cornea providing an interface with the aqueous humor; (4) a pupil to limit the amount of light; (5) an IOL comprising a material having a refractive index and an anterior surface configured to refract light incident on the IOL; (6) an observer located between about 0.5 m and about 2.0 m away from the anterior surface of the IOL; and (7) a constructed physical eye model. The simulation tool is configured to provide controls that can vary the position of the anterior surface of the IOL with respect to the posterior surface of the cornea. The simulation tool is configured such that the observer can have a range of pupil sizes (e.g., pupil size of 3 mm, 5 mm, 8 mm, 10 mm, etc.). This may help in determining whether the peak intensity of the reflected light is in the observer's pupillary plane.

Using the simulation tool, the effect of radius of curvature of the anterior surface of the IOL, the effective lens position (ELP) with respect to the posterior surface of the cornea, the location of the observer, the wavelength of incident light and/or the corneal radius on pupillary reflection was studied.

To study the effect of radius of curvature of the anterior surface of the IOL on the intensity of light reflected from the IOL, light from a collimated source of light was made incident on the IOL. The IOL was considered to comprise Sensar® which has an index of refraction of about 1.47. FIG. 1 illustrates a graph of the intensity of light reflected from the anterior surface of the IOL as detected by an observer located at a distance of 1 m from the anterior surface for different values of the radius of curvature of the anterior surface. The intensity of light reflected from the anterior surface is represented on a logarithmic scale. It is noted from FIG. 1 that the intensity of light reflected from the anterior surface of the IOL peaks for radius of curvature of the anterior surface between about 24 mm and about 32 mm. The maximum of the intensity of light reflected from the IOL occurs at a radius of curvature of the anterior surface of about 26 mm. It is further noted that the intensity of light reflected from an IOL having an anterior surface with a radius of curvature of about 26 mm is 1000 times higher than the intensity of light reflected from an IOL having an anterior surface with a radius of curvature of about 32 mm or 24 mm.

Based on FIG. 1, IOLs with an anterior surface having a radius of curvature greater than or equal to about 35 mm or less than or equal to about 20 mm have reduced pupillary reflections. For example, intensity pupillary reflections from IOLs with an anterior surface having a radius of curvature greater than or equal to about 35 mm and less than or equal to about 55 mm, greater than or equal to about 40 mm and less than or equal to about 50 mm, greater than or equal to about 42 mm and less than or equal to about 48 mm, or a value in these ranges or sub-ranges can be reduced as compared to from IOLs with an anterior surface having a radius of curvature between about 24 mm and about 32 mm. The intensity of pupillary reflections from IOLs with an anterior surface having a radius of curvature greater than or equal to about 35 mm and less than or equal to about 55 mm, greater than or equal to about 40 mm and less than or equal to about 50 mm, greater than or equal to about 42 mm and less than or equal to about 48 mm, or a value in these ranges or sub-ranges can be below a detection threshold of an observer. As another example, intensity pupillary reflections from IOLs with an anterior surface having a radius of curvature less than or equal to about 20 mm, less than or equal to about 19 mm, less than or equal to about 15 mm, less than or equal to about 10 mm, less than or equal to about 5 mm, or a value in these ranges or sub-ranges can be reduced as compared to from IOLs with an anterior surface having a radius of curvature between about 24 mm and about 32 mm. The intensity of pupillary reflections from IOLs with an anterior surface having a radius of curvature less than or equal to about 20 mm, less than or equal to about 19 mm, less than or equal to about 15 mm, less than or equal to about 10 mm, less than or equal to about 5 mm, or a value in these ranges or sub-ranges can be below a detection threshold of an observer. From FIG. 1, it can be concluded that if the anterior surface of the IOL has a radius of curvature outside of a range between about 24 mm and about 32 mm, then the intensity of light reflected from the IOL can be below a detection threshold of an observer.

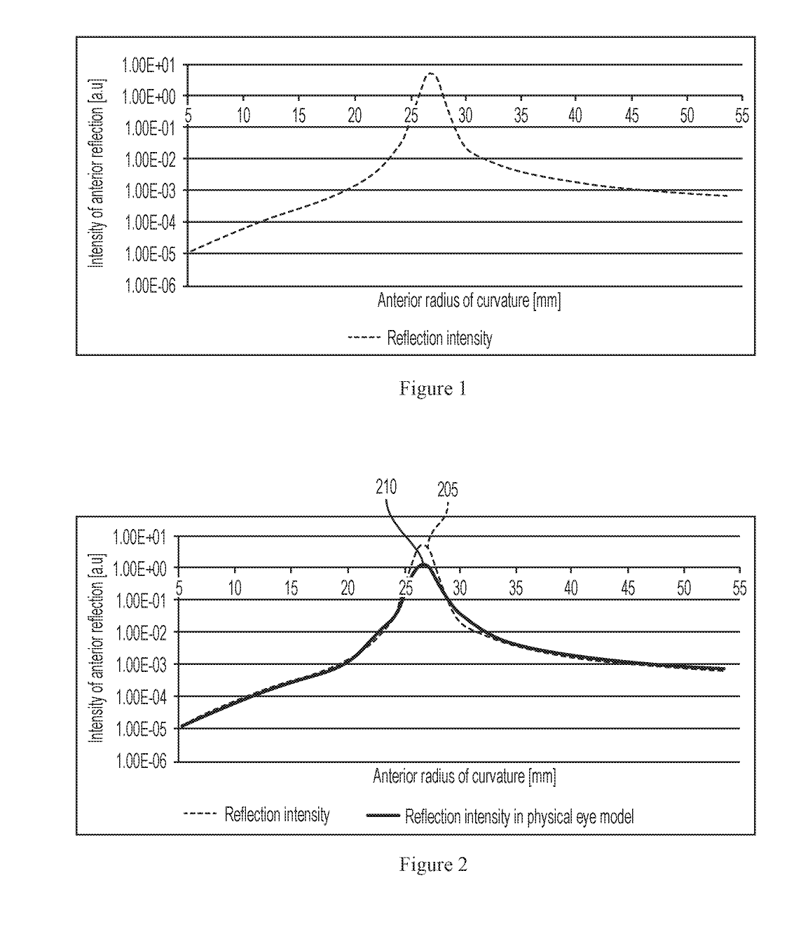

FIG. 2 is a graph illustrating a comparison between the intensity of light reflected from an IOL placed in a constructed physical eye model and the intensity of light reflected from the IOL placed in a real eye. Curve 205 is the intensity of light reflected from the IOL placed in the constructed physical eye model and curve 210 is the intensity of light reflected from an IOL placed in a real eye. As noted from FIG. 2, the intensity of light reflected from the IOL included in the real eye also peaks for values of radius of curvature of the anterior surface of the IOL between about 24 mm and about 32 mm similar to the intensity of light reflected from the IOL placed in the constructed physical eye model. FIG. 2 also demonstrates the validity of the constructed physical eye model.

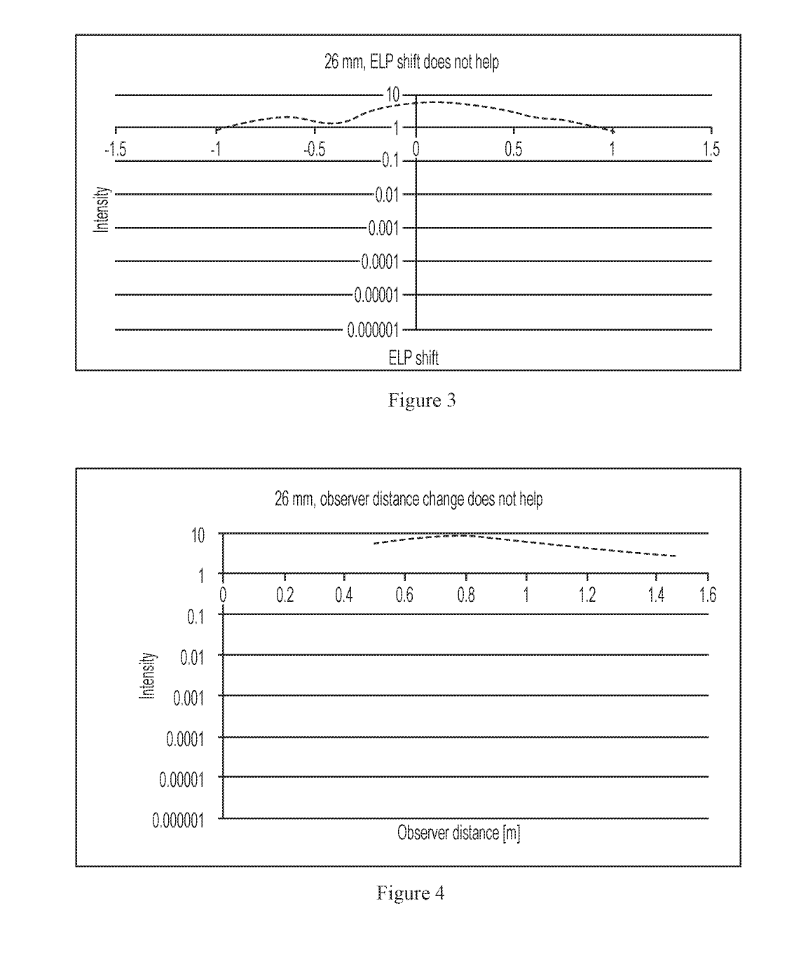

The simulation tool was used to study the effect of various factors, such as, for example, (i) effective lens position (ELP) of the anterior surface of the IOL with respect to the cornea, (ii) distance of the observer from the anterior surface of the IOL, (iii) wavelength of incident ambient light and (iv) the corneal radius on the intensity of light reflected from the he IOL. To study the effect of the various factors discussed above, the radius of curvature of the anterior surface of the IOL was set to 26 mm in the simulation tool—which corresponds to the radius of curvature of the anterior surface of the IOL at which light reflected from the IOL has maximum intensity as noted from FIGS. 1 and 2. FIG. 3 illustrates the variation in the intensity of light reflected from an IOL having a radius of curvature of 26 mm as a function of the effective lens position (ELP). As noted from FIG. 3, the intensity of light reflected from the IOL does not reduce significantly (e.g., the variation of the intensity of light reflected from the IOL is within an order of magnitude) as the position of the IOL is varied between about ±1 mm from an original position of about 4.5 mm from the cornea. Thus, the ELP with respect to the cornea does not appear to significantly affect the intensity of light reflected from the IOL. The intensity of light reflected from the IOL appears to be independent of the ELP. It should be noted that pushing the IOL further away from the cornea towards the retina may result in a shift of which anterior radius of curvature gives the highest reflection. For example, the intensity of light reflected from the IOL can occur at a value of radius of curvature of the anterior surface that is different from 26 mm. However, the amount by which the peak of the intensity of reflected light is shifted may be in the range of about 2 mm or less.

FIG. 4 illustrates the variation in the intensity of light reflected from an IOL having a radius of curvature of 26 mm as a function of the position of an observer. As noted from FIG. 4, the intensity of light reflected from the IOL does not reduce significantly (e.g., the variation of the intensity of light reflected from the IOL is within an order of magnitude) as the position of the observer is varied between about 0.5 m and about 2.0 m from the anterior surface of the IOL. Thus, the position of the observer with respect to the anterior surface of the IOL does not appear to significantly affect the intensity of light reflected from the IOL. The intensity of light reflected from the IOL appears to be independent of the position of the observer.

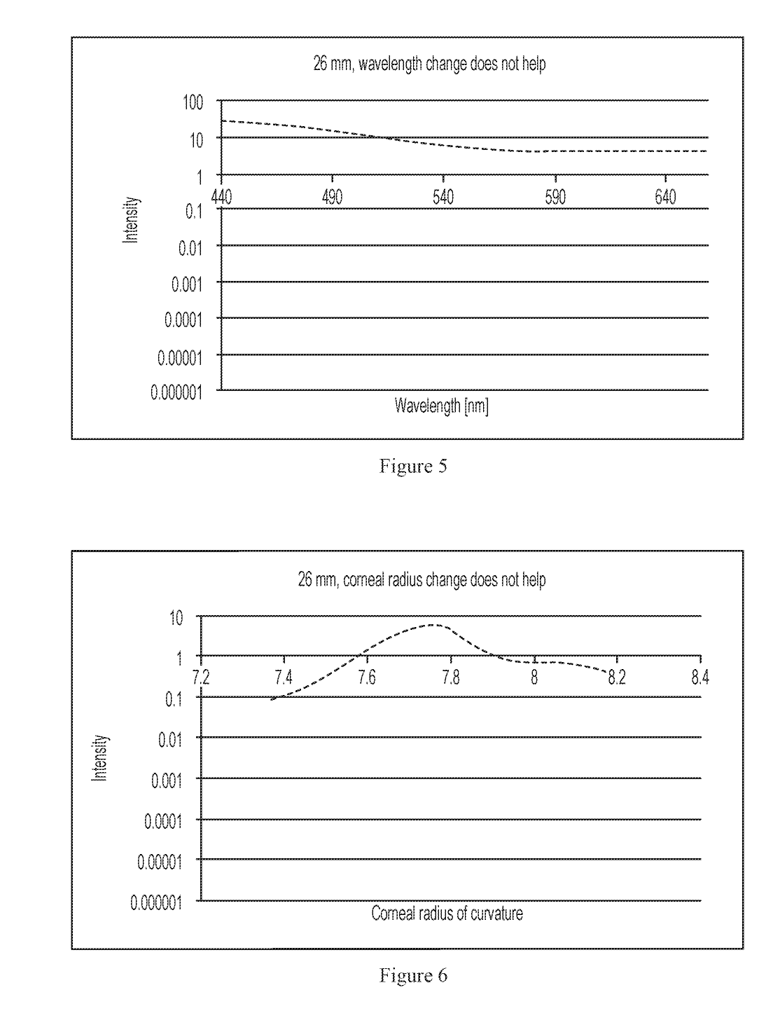

FIG. 5 illustrates the variation in the intensity of light reflected from an IOL having a radius of curvature of 26 mm as a function of the wavelength of incident light in visible wavelength range (e.g., between about 440 nm and about 650 nm). As noted from FIG. 4, the intensity of light reflected from the IOL does not reduce significantly (e.g., the variation of the intensity of light reflected from the IOL is within an order of magnitude) as the wavelength of incident light is varied. Thus, the wavelength of incident light does not appear to significantly affect the intensity of light reflected from the IOL. The intensity of light reflected from the IOL appears to be independent of the wavelength of incident light.

FIG. 6 illustrates the variation in the intensity of light reflected from an IOL having a radius of curvature of 26 mm as a function of the corneal radius. As noted from FIG. 6, the intensity of light reflected from the IOL does not reduce significantly (e.g., the variation of the intensity of light reflected from the IOL is within an order of magnitude) as the corneal radius is varied between about 7.6 mm and about 8.1 mm. Thus, a variation of corneal radius between about 7.6 mm and about 8.1 mm does not appear to significantly affect the intensity of light reflected from the IOL. The intensity of light reflected from the IOL appears to be independent of variation of corneal radius in the range between about 7.6 mm and about 8.1 mm. The average corneal radius of a human eye is about 7.8 mm. Thus, the intensity of light reflected from the IOL appears to be independent of variation of corneal radius in a population of human eyes.

Based on the above study, it appears that the radius of curvature of the anterior surface of the IOL can influence the variation in intensity of light reflected from the IOL to a greater extent than the effective lens position, the position of the observer, the wavelength of incident light and/or the corneal radius. Based on the study it can be predicted that the intensity of light reflected from the IOL having an anterior surface with a radius of curvature of about 26 mm will be greater than the intensity of light reflected from an IOL having an anterior surface with a radius of curvature less than about 24 mm or greater than about 32 mm. The predictions of the study were verified using first IOL having an anterior surface with a radius of curvature of about 1000 mm, a second IOL having an anterior surface with a radius of curvature of about 26 mm and a third IOL having an anterior surface with a radius of curvature of about 12 mm in the constructed physical eye model. In accordance with the current state of art, it is believed that the intensity of light reflected from a flatter lens (e.g., having an anterior surface with a larger radius of curvature) is greater than the intensity of light reflected from a curved lens (e.g., having an anterior surface with a smaller radius of curvature). However, the results of the study described herein show that the intensity of light reflected from a flatter lens, such as, for example, a lens having an anterior surface with a radius of curvature greater than 42 mm (e.g., 1000 mm) is smaller than the intensity of light reflected from a curved lens, such as, for example, a lens having an anterior surface with a radius of curvature between about 24 mm and about 32 mm.

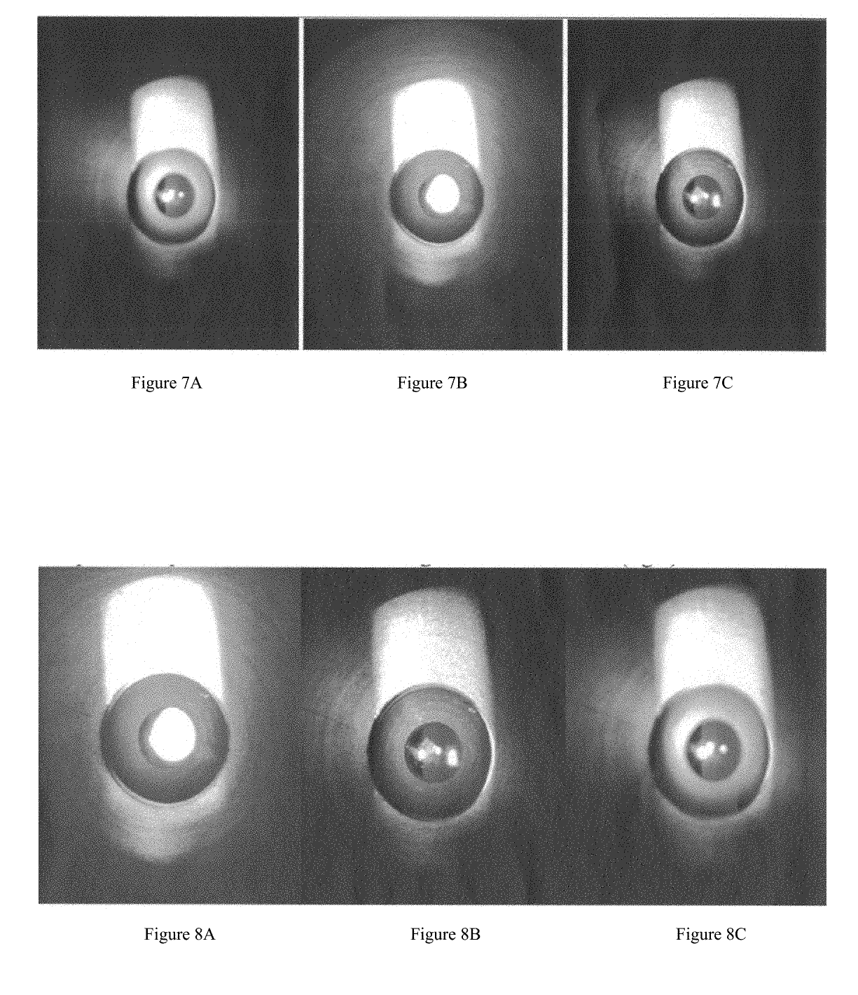

FIG. 7A is a photograph depicting the reflection of light from the first IOL having an anterior surface with a radius of curvature of about 1000 mm. FIG. 7B is a photograph depicting the reflection of light from the second IOL having an anterior surface with a radius of curvature of about 26 mm. FIG. 7C is a photograph depicting the reflection of light from the third IOL having an anterior surface with a radius of curvature of about 12 mm. It is noted that although the first IOL and the third IOL having an anterior surface with a radius of curvature of about 1000 mm and about 12 mm respectively exhibit reflections that originate in the cornea and the back window of the constructed physical eye model, the intensity of reflection from the first IOL and the third IOLs is dwarfed by the intensity of reflection from the second IOL. FIGS. 7A-7C appear to confirm the predictions of the study—that the intensity of light reflected from the second IOL having an anterior surface with a radius of curvature of about 26 mm is greater than the intensity of light reflected from the first IOL having an anterior surface with a radius of curvature of about 1000 mm or the third IOL having an anterior surface with a radius of curvature of about 12 mm.

The simulation tool was used to obtain some predictions regarding the intensity of light reflected from a set of commercial embodiments of IOLs. Assuming that the Acrysof IQ® lens was biconvex, the simulation tool predicted that the intensity of light reflected from the Acrysof IQ® IOL with 16.5 D optical power would be greater than Acrysof IQ® IOLs with other optical power. Accordingly, the simulation tool predicted that the Acrysof IQ® IOL with 16.5 D optical power would have the worst reflectivity among other models of Acrysof IQ® IOLs. To verify the predictions of the simulation tool, an embodiment of an Acrysof IQ® IOL with 15 D optical power, an embodiment of an Acrysof IQ® IOL with 25 D optical power and an embodiment of a Tecnis® IOL with 15 D optical power were tested with the constructed physical eye model. The simulation tool predicted that the Acrysof IQ® IOL with 15 D optical power would have a reflectivity of 0.2 as a result of the design/geometry of the IOL which was elevated to 0.5 due to increased reflectivity of the Acrysof material. Although, the reflectivity of the Acrysof IQ® IOL with 15 D optical power predicted by the simulation tool was less than the reflectivity of the Acrysof IQ® IOL with 16.5 D optical power, the reflectivity was higher than the predicted reflectivity of other Acrysof IQ® IOL models. Based on the assumption that the Acrysof IQ® IOL with 15 D optical power and the Acrysof IQ® IOL with 25 D optical power were equiconvex lenses, the simulation tool predicted that the radius of curvature of the Acrysof IQ® IOL with 15 D optical power and the Acrysof IQ® IOL with 25 D optical power were 29 mm and about 20 mm respectively.

The different embodiments of commercially available IOLs were included in the constructed physical eye model to test the predictions of the simulation tool. FIG. 8A is a photograph depicting the reflection of light from Acrysof IQ® IOL with 15 D optical power. FIG. 8B is a photograph depicting the reflection of light from Acrysof IQ® IOL with 25 D optical power. FIG. 8C is a photograph depicting the reflection of light from Tecnis® IOL with 15 D optical power of a commercially available IOL. It is noted that the intensity of light reflected from the Acrysof IQ® IOL with 15 D optical power (presumed to have an anterior surface with a radius of curvature equal to about 29 mm based on the assumption that the lens is equiconvex) is greater than the intensity of light reflected from the Acrysof IQ® IOL with 25 D optical power (presumed to have an anterior surface with a radius of curvature equal to about 20 mm based on the assumption that the lens is equiconvex). It is further noted that the intensity of light reflected from the Acrysof IQ® IOL with 15 D optical power (presumed to have an anterior surface with a radius of curvature equal to about 29 mm based on the assumption that the lens is equiconvex) is greater than the intensity of light reflected from the Tecnis® IOL with 15 D optical power (having an anterior surface with a radius of curvature equal to about 18 mm). From FIGS. 8A-8C it can be concluded that the difference in the reflectivity of the Acrysof IQ® IOL with 15 D optical power and the Acrysof IQ® IOL with 25 D optical power can be attributed to the radius of curvature of the anterior surface while the difference in the reflectivity of the Acrysof IQ® IOL with 15 D optical power and the Tecnis® IOL with 15 D optical power can be attributed to the difference in the refractive indices—1.55 for Acrysof® and 1.47 for Sensar®, the material of the Tecnis® IOL.

It is noted that the radius of curvature at which the reflection intensity peak has a maximum can depend on the refractive index of the material of the IOL. However, the contribution to the intensity of reflection from the refractive index difference between the IOL and the surrounding medium is much less as compared to the contribution to the intensity of reflection from the geometry of the anterior surface. Thus, the radius of curvature at which the reflection intensity peak has a maximum may shift by about ±2.0 mm (e.g., from 26 mm to about 28 mm or about 24 mm) for IOLs with materials having refractive index different from 1.47.

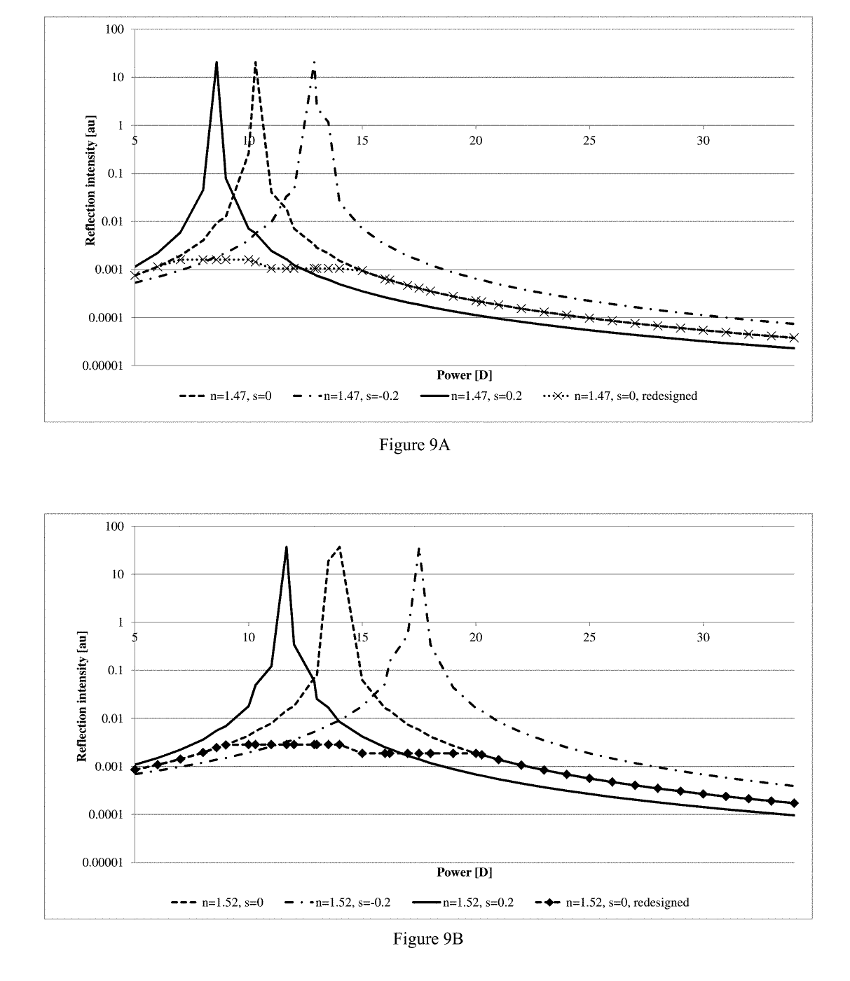

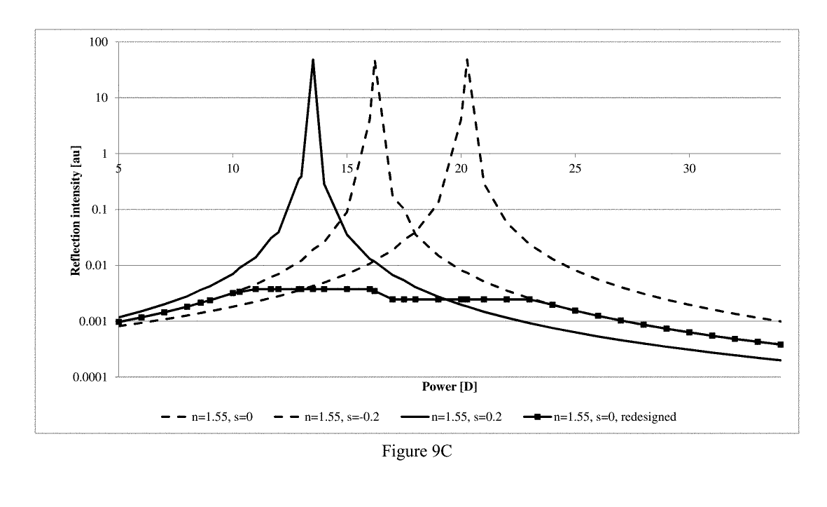

It is further noted that if the IOL comprises a material with a high index of refraction (e.g., Acrysof® which has a refractive index of 1.55), the design space may be constrained if a certain range of values for the radius of curvature of the anterior surface should be avoided in order to reduce intensity of light reflected from the IOL. In particular, shape factors around 0 would result in a high reflectivity for the most common diopter powers, which are around 20 D. The intensity of light reflected from embodiments of IOLs having different shape factors and different materials as a function of optical power was studied using the simulation tool described herein. FIG. 9A is a graph of the intensity of light reflected from various embodiments of IOLs having different shape factors and comprising a material having refractive index of 1.47 (e.g., Sensar®) for different optical powers. FIG. 9B is a graph of the intensity of light reflected from various embodiments of IOLs having different shape factors and comprising a material having refractive index of 1.52 (e.g., AF-1®) for different optical powers. FIG. 9C is a graph of the intensity of light reflected from various embodiments of IOLs having different shape factors and comprising a material having refractive index of 1.55 (e.g., Acrysof®) for different optical powers.

It is noted from FIG. 9A that the intensity of light reflected from an embodiment of an IOL comprising a material having a refractive index of 1.47, such as, for example SENSAR® and having a shape factor of 0.2 determined without constraining the radius of curvature of the anterior surface to be either less than 24 mm (e.g., less than or equal to about 19 mm) or greater than about 35 mm (e.g., greater than or equal to about 42 mm) peaks for optical power between about 6 and 11 Diopter and attains a maxima at an optical power of about 9 Diopter. The intensity of light reflected from an embodiment of an IOL comprising a material having a refractive index of 1.47, such as, for example SENSAR® and having a shape factor of −0.2 determined without constraining the radius of curvature of the anterior surface to be either less than 24 mm (e.g., less than or equal to about 19 mm) or greater than about 35 mm (e.g., greater than or equal to about 42 mm) peaks for optical power between about 9 and 17 Diopter and attains a maxima at an optical power of about 13 Diopter. The intensity of light reflected from an embodiment of an IOL comprising a material having a refractive index of 1.47, such as, for example SENSAR® and having a shape factor of 0 determined without constraining the radius of curvature of the anterior surface to be either less than 24 mm (e.g., less than or equal to about 19 mm) or greater than about 35 mm (e.g., greater than or equal to about 42 mm) peaks for optical power between about 7 and 15 Diopter and attains a maxima at an optical power of about 10 Diopter.

It is further noted from FIG. 9A that the intensity of reflection in the vicinity of the maxima is about 3-4 orders of magnitude greater than the intensity of reflection outside the peak region for all shape factors 0, 0.2 and −0.2 when the radius of curvature of the anterior surface is not constrained to be either less than 24 mm (e.g., less than or equal to about 19 mm) or greater than about 35 mm (e.g., greater than or equal to about 42 mm). However, in accordance with the results of the study described herein, if the shape factor of the IOL is determined by constraining the radius of curvature of the anterior surface to be either less than 24 mm (e.g., less than or equal to about 19 mm) or greater than about 35 mm (e.g., greater than or equal to about 42 mm), then the peaks in the intensity of light reflected from the IOL can be reduced. For example, in FIG. 9A, the embodiment of an IOL comprising a material having a refractive index of 1.47, such as, for example SENSAR® and having a shape factor of 0 is redesigned such that the radius of curvature of the anterior surface is either less than 24 mm (e.g., less than or equal to about 19 mm) or greater than about 35 mm (e.g., greater than or equal to about 42 mm) while maintaining the shape factor of 0, does not exhibit any peaks in the intensity of light reflected from the IOL. The intensity of light reflected from the redesigned embodiment of an IOL comprising a material having a refractive index of 1.47, such as, for example SENSAR® and having a shape factor of 0 is within two orders of magnitude for a range of optical powers between about 5 Diopter and about 34 Diopter.

Without subscribing to any particular theory, the shape factor (X) of a lens is calculated using equation 2 below:

$$X = ( \frac{r 2 + r 1}{r 2 - r 1} ) \qquad ( 2 )$$

where r2 is the radius of curvature of the posterior surface and r1 is the radius of curvature of the anterior surface. A shape factor (X) of 0.2 indicates that the anterior surface contributes about 60% of the optical power and the posterior surface contributes about 40% of the optical power. A shape factor (X) of −0.2 indicates that the anterior surface contributes about 40% of the optical power and the posterior surface contributes about 60% of the optical power. An equi-convex lens has a shape factor (X) of 0 indicating that the anterior surface and the posterior surface each contribute about 50% of the optical power.

FIGS. 9B and 9C indicate that the embodiments of IOLs comprising material having refractive index 1.52 and 1.55 respectively can be similarly redesigned by constraining the radius of curvature of the anterior surface to be either less than 24 mm (e.g., less than or equal to about 19 mm) or greater than about 35 mm (e.g., greater than or equal to about 42 mm). For example, referring to FIG. 9B, the embodiment of an IOL comprising a material having a refractive index of 1.52, such as, for example AF-1® and having a shape factor of 0 is redesigned such that the radius of curvature of the anterior surface is either less than 24 mm (e.g., less than or equal to about 19 mm) or greater than about 35 mm (e.g., greater than or equal to about 42 mm) while maintaining the shape factor of 0, does not exhibit any peaks in the intensity of light reflected from the IOL. As another example, referring to FIG. 9C, the embodiment of an IOL comprising a material having a refractive index of 1.55, such as, for example Acrysof® and having a shape factor of 0 is redesigned such that the radius of curvature of the anterior surface is either less than 24 mm (e.g., less than or equal to about 19 mm) or greater than about 35 mm (e.g., greater than or equal to about 42 mm) while maintaining the shape factor of 0, does not exhibit any peaks in the intensity of light reflected from the IOL.

### Example Method of Designing an IOL Having Reduced Pupillary Reflections

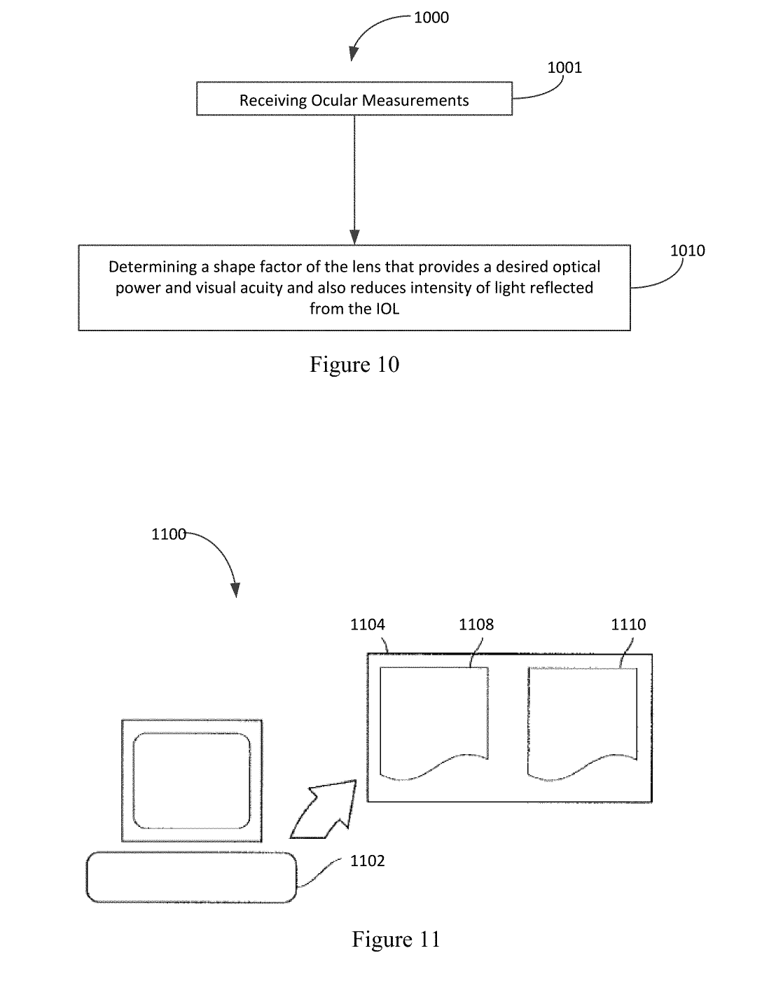

An example method of designing an IOL to have reduced pupillary reflections is illustrated in FIG. 10. The method 1000 includes receiving ocular measurements for a patient as shown in block 1001. The ocular measurements can be obtained by an ophthalmologist using instruments such as a COAS or a biometer which are currently available in ophthalmology practice. The ocular measurements can include axial length of the eye, corneal power, refractive power that provides visual acuity for central vision, intraocular pressure, peripheral refractive errors measured by a visual fields test and any other measurements that can be used to characterize a patient's visual acuity for central vision as well as peripheral vision.

The method 1000 further includes determining a shape factor of the IOL that provides a desired power and visual acuity while also reducing the intensity of light reflected from the IOL as shown in block 1010. In various embodiments, the shape factor of the IOL is determined cy constraining the radius of curvature to be outside of a range between about 24 mm and about 32 mm, between about 20 mm and about 35 mm or between about 20 mm and about 40 mm. In some embodiments, the shape factor can be determined by selecting a radius of curvature of the anterior surface from a first range of values for a first range of optical powers and selecting a radius of curvature of the anterior surface from a second range of values for a second range of optical powers. The first and the second range of values can be non-overlapping. For example, the shape factor can be determined by selecting a radius of curvature of the anterior surface to have a value in a range between about 35 mm and about 2000 mm (e.g., between about 36 mm and about 1000 mm, between about 38 mm and about 500 mm, between about 39 mm and about 100 mm, between about 40 mm and about 75 mm, between about 42 mm and about 50 mm, or any value in these ranges or sub-ranges including the end points) for optical power in the range between about 0 and about 25 Diopter (e.g., between about 1 Diopter and about 25 Diopter, between about 5 Diopter and about 24 Diopter, between about 10 Diopter and about 22 Diopter, between about 15 Diopter and about 21 Diopter, between about 17 Diopter and about 22 Diopter, between about 19 Diopter and about 21 Diopter or any value in these ranges or sub-ranges including the end points).

The shape factor can be determined by selecting a radius of curvature of the anterior surface to have a value in a range between about 1 mm and about 23 mm (e.g., between about 2 mm and about 22 mm, between about 3 mm and about 21 mm, between about 5 mm and about 20 mm, between about 6 mm and about 19 mm, between about 7 mm and about 18 mm, less than 19 mm, less than 17 mm, less than 15 mm or any value in these ranges or sub-ranges including the end points) for optical power in the range between about 20 and about 34 Diopter (e.g., between about 21 Diopter and about 33 Diopter, between about 22 Diopter and about 32 Diopter, between about 25 Diopter and about 30 Diopter, between about 26 Diopter and about 29 Diopter, or any value in these ranges or sub-ranges including the end points).

The method of designing an IOL that reduces pupillary reflection can be implemented by a computer system 1100 illustrated in FIG. 11. The system includes a processor 1102 and a computer readable memory 1104 coupled to the processor 1102. The computer readable memory 1104 has stored therein an array of ordered values 1108 and sequences of instructions 1110 which, when executed by the processor 402, cause the processor 1102 to perform certain functions or execute certain modules. For example, a module can be executed that is configured to selecting an ophthalmic lens or an optical power thereof that would provide visual acuity for central vision and iteratively adjust various parameters of the lens including but not limited to radius of curvature of the anterior surface of the IOL to reduce intensity of light reflected from the IOL.

The array of ordered values 1108 may comprise, for example, one or more ocular dimensions of an eye or plurality of eyes from a database, a desired refractive outcome, parameters of an eye model based on one or more characteristics of at least one eye, and data related to an IOL or set of IOLs such as a power, an aspheric profile, and/or a lens plane. In some embodiments, the sequence of instructions 1110 includes determining a position of an IOL, performing one or more calculations to determine a predicted refractive outcome based on an eye model and a ray tracing algorithm, comparing a predicted refractive outcome to a desired refractive outcome, and based on the comparison, repeating the calculation with an IOL having at least one of a different power, different design, and/or a different IOL location.

The computer system 1100 may be a general purpose desktop or laptop computer or may comprise hardware specifically configured performing the desired calculations. In some embodiments, the computer system 1100 is configured to be electronically coupled to another device such as a phacoemulsification console or one or more instruments for obtaining measurements of an eye or a plurality of eyes. In other embodiments, the computer system 1100 is a handheld device that may be adapted to be electronically coupled to one of the devices just listed. In yet other embodiments, the computer system 1100 is, or is part of, refractive planner configured to provide one or more suitable intraocular lenses for implantation based on physical, structural, and/or geometric characteristics of an eye, and based on other characteristics of a patient or patient history, such as the age of a patient, medical history, history of ocular procedures, life preferences, and the like.

In certain embodiments, the system 1100 includes or is part of a phacoemulsification system, laser treatment system, optical diagnostic instrument (e.g, autorefractor, aberrometer, and/or corneal topographer, or the like). For example, the computer readable memory 1104 may additionally contain instructions for controlling the handpiece of a phacoemulsification system or similar surgical system. Additionally or alternatively, the computer readable memory 1104 may comprise instructions for controlling or exchanging data with an autorefractor, aberrometer, tomographer, and/or topographer, or the like.

In some embodiments, the system 1100 includes or is part of a refractive planner. The refractive planner may be a system for determining one or more treatment options for a subject based on such parameters as patient age, family history, vision preferences (e.g., near, intermediate, distant vision), activity type/level, past surgical procedures.

### CONCLUSION

The above presents a description of the best mode contemplated of carrying out the concepts disclosed herein, and of the manner and process of making and using it, in such full, clear, concise, and exact terms as to enable any person skilled in the art to which it pertains to make and use the concepts described herein. The systems, methods and devices disclosed herein are, however, susceptible to modifications and alternate constructions from that discussed above which are fully equivalent. Consequently, it is not the intention to limit the scope of this disclosure to the particular embodiments disclosed. On the contrary, the intention is to cover modifications and alternate constructions coming within the spirit and scope of the present disclosure as generally expressed by the following claims, which particularly point out and distinctly claim the subject matter of the implementations described herein.

Although embodiments have been described and pictured in an example form with a certain degree of particularity, it should be understood that the present disclosure has been made by way of example, and that numerous changes in the details of construction and combination and arrangement of parts and steps may be made without departing from the spirit and scope of the disclosure as set forth in the claims hereinafter.

As used herein, the term “processor” refers broadly to any suitable device, logical block, module, circuit, or combination of elements for executing instructions. For example, the processor 1102 can include any conventional general purpose single- or multi-chip microprocessor such as a Pentium® processor, a MIPS® processor, a Power PC® processor, AMD® processor, ARM processor, or an ALPHA® processor. In addition, the processor 302 can include any conventional special purpose microprocessor such as a digital signal processor. The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. Processor 1102 can be implemented as a combination of computing devices, e.g., a combination of a DSP and a microprocessor, a plurality of microprocessors, one or more microprocessors in conjunction with a DSP core, or any other such configuration.

Computer readable memory 1104 can refer to electronic circuitry that allows information, typically computer or digital data, to be stored and retrieved. Computer readable memory 404 can refer to external devices or systems, for example, disk drives or solid state drives. Computer readable memory 1104 can also refer to fast semiconductor storage (chips), for example, Random Access Memory (RAM) or various forms of Read Only Memory (ROM), which are directly connected to the communication bus or the processor 1102. Other types of memory include bubble memory and core memory. Computer readable memory 1104 can be physical hardware configured to store information in a non-transitory medium.

Methods and processes described herein may be embodied in, and partially or fully automated via, software code modules executed by one or more general and/or special purpose computers. The word “module” can refer to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may comprise connected logic units, such as gates and flip-flops, and/or may comprised programmable units, such as programmable gate arrays, application specific integrated circuits, and/or processors. The modules described herein can be implemented as software modules, but also may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with users, operators, other systems, components, programs, and so forth.

What is claimed:

1. A plurality of intraocular lenses configured to provide an optical power between about 5 Diopter and about 34 Diopter at a predefined increment there between, each lens of the plurality of lenses comprising a convex anterior surface configured to receive ambient light refracted by a cornea, a posterior surface opposite the anterior surface, and a shape factor, wherein the shape factor of each lens is configured such that the magnitude of intensity of light reflected from any lens of the plurality of intraocular lenses is within two orders of magnitude of the intensity of light reflected from any other lens of the plurality of intraocular lenses.

2. The plurality of intraocular lenses of claim 1, wherein a ratio between the magnitude of intensity of light reflected from any lens of the plurality of intraocular lenses and a magnitude of the intensity of light reflected from any other lens of the plurality of intraocular lenses is less than 100.

3. The plurality of intraocular lenses of claim 1, wherein a difference between the magnitude of intensity of light reflected from any lens of the plurality of intraocular lenses and a magnitude of the intensity of light reflected from any other lens of the plurality of intraocular lenses is less than 1000%.

4. The plurality of intraocular lenses of claim 1, wherein the anterior surface of each of the plurality of intraocular lenses has a radius of curvature less than about 24 mm or greater than about 32 mm.

5. The plurality of intraocular lenses of claim 4, wherein the radius of curvature of the anterior surface of each of the plurality of intraocular lenses is less than about 20 mm or greater than about 36 mm.

6. The plurality of intraocular lenses of claim 5, wherein the radius of curvature of the anterior surface of each of the plurality of intraocular lenses is less than or equal to about 19 mm or greater than or equal to about 42 mm.

7. The plurality of intraocular lenses of claim 1, wherein no lens of the plurality of intraocular lenses has an anterior surface with a radius of curvature between about 24 mm and about 32 mm.

8. The plurality of intraocular lenses of claim 1, wherein the plurality of intraocular lenses are configured to provide optical power between about 12 Diopter and about 30 Diopter.

9. The plurality of intraocular lenses of claim 1, wherein the plurality of intraocular lenses are configured to provide optical power between about 17 Diopter and about 25 Diopter.

10. The plurality of intraocular lenses of claim 1, wherein the plurality of intraocular lenses are configured to provide optical power between about 19 Diopter and about 21 Diopter.

11. A method of designing an intraocular lens, the method comprising:
obtaining at least one physical or optical characteristic of the patient's eye using a diagnostic instrument; and
determining a shape factor of an intraocular lens that provides a desired optical power, wherein the intraocular lens has a convex anterior surface having a radius of curvature that is outside of a range between 24 mm and 32 mm for any optical power.

12. The method of claim 12, wherein the radius of curvature of the anterior surface is less than or equal to about 20 mm for optical power greater than or equal to about 26 Diopter.

13. The method of claim 12, wherein the wherein the radius of curvature of the anterior surface is greater than or equal to about 35 mm for optical power less than about 26 Diopter.

14. A method of designing an intraocular lens, the method comprising:
obtaining at least one physical or optical characteristic of the patient's eye using a diagnostic instrument; and
determining a shape factor of an intraocular lens that provides a desired optical power, wherein the intraocular lens has a convex anterior surface,
wherein determining the shape factor comprises selecting a value for a radius of curvature of an anterior surface of the intraocular lens from a first range of values for a first range of optical powers and a second range of values for a second range of optical powers to reduce a peak intensity of reflected ambient light over a range of clinical optical powers including the first and the second range of optical powers, and
wherein the first range of values and the second range of values are non-overlapping.

15. The method of claim 15, wherein values of radius of curvature in the first range of values are less than 24 mm and values of optical powers in the first range of power are greater than 25 Diopter.

16. The method of claim 15, wherein values of radius of curvature in the second range of values are greater than 32 mm and values of optical powers in the second range of power are less than 25 Diopter.
